(19) United States
(12) Patent Application Publication
(10) Pub. No.: US 2019/0200857 A1
(43) Pub. Date: Jul. 4, 2019

(54) MULTIPLE OFF-AXIS CHANNEL OPTICAL IMAGING DEVICE UTILIZING UPSIDE-DOWN PYRAMIDAL CONFIGURATION

(71) Applicant: BROADSPOT IMAGING CORP, Richmond, CA (US)

(72) Inventors: Tushar M. RANCHOD, Berkeley, CA (US); Benjamin A. JACOBSON, Santa Barbara, CA (US); Clark PENTICO, Simi Valley, CA (US); Andre E. ADAMS, Tiburon, CA (US); Brendan Hugo HAMEL-BISSELL, San Francisco, CA (US)

(73) Assignee: BROADSPOT IMAGING CORP, Richmond, CA (US)

(21) Appl. No.: 16/217,750

(22) Filed: Dec. 12, 2018

Related U.S. Application Data
(60) Provisional application No. 62/611,061, filed on Dec. 28, 2017.

(51) Int. Cl. A61B 3/10; H04N 5/247; H04N 5/225; G06T 3/40; A61B 3/14; A61B 3/12

(52) U.S. Cl. CPC A61B 3/1025; H04N 5/247; A61B 3/12; G06T 3/4038; A61B 3/14; H04N 5/2251

(57) ABSTRACT

An optical imaging device may include a support structure and multiple imaging channels, where each of the imaging channels includes a discrete optical imaging pathway disposed within the support structure. Additionally, the imaging channels may be aimed at different angles relative to each other such that each optical imaging pathway is directed through a pupil of the eye towards corresponding partially overlapping regions of a retina.

### FIELD

The application relates generally to a multiple off-axis channel optical imaging device utilizing upside-down pyramidal configuration.

### BACKGROUND

Ocular imaging is commonly used both to screen for diseases and to document findings discovered during clinical examination of the eye. Specifically, documentation and analysis of optical imaging may be relevant to comprehensive eye examinations and full evaluations of current conditions, treatment, and/or early prevention of various eye conditions and diseases. Component configurations of optical imaging devices may affect a clearance distance relative to the facial structure of patients.

The subject matter claimed herein is not limited to embodiments that solve any disadvantages or that operate only in environments such as those described above. Rather, this background is only provided to illustrate one example technology area where some embodiments described herein may be practiced.

### SUMMARY

Embodiments of the present disclosure discuss an optical imaging device. The optical imaging device may include a support structure and multiple imaging channels, where each of the imaging channels includes a discrete optical imaging pathway disposed within the support structure. In these embodiments, the imaging channels may be aimed at different angles relative to each other such that each optical imaging pathway is directed through a pupil of the eye towards corresponding partially overlapping regions of a retina.

The objects and advantages of the embodiments will be realized and achieved at least by the elements, features, and combinations particularly pointed out in the claims.

Both the foregoing general description and the following detailed description are given as examples and are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1A illustrates a cross-sectional side view of an eye, including an example optical imaging pathway for imaging the eye;

FIG. 1B illustrates another cross-sectional side view of the eye of FIG. 1A, including multiple example optical imaging pathways for imaging the eye;

FIG. 1C illustrates a cross-sectional front view of the eye of FIG. 1A, including multiple overlapping imaging regions of the eye;

FIG. 2A illustrates a cross-sectional side view of a device for imaging the eye in FIG. 1A, relative to facial features;

FIG. 2B illustrates a cross-sectional top view of a device for imaging the eye in FIG. 1A, relative to facial features;

FIG. 3 illustrates a cross-sectional front view of a device for imaging the eye in FIG. 1A, relative to facial features; and

FIG. 4 illustrates an example system that may be used in multiple off-axis channel imaging of the eye.

### DESCRIPTION OF EMBODIMENTS

In some embodiments of the present disclosure, imaging channels each with at least one unique imaging pathway, may approach the eye at different angles. The respective imaging pathways may cross each other within the plane of the iris of the human eye, or within the space between the cornea and the mid-vitreous cavity. In these or other embodiments, none of the imaging channels may be coaxial with a central axis of the eye. However, in some embodiments, at least one imaging channel may be coaxial with the central axis of the eye. The imaging channels may image different but partially overlapping regions of the eye such that the resulting images can be stitched into a single composite optical image with a combined area greater than any constituent image and in such a way that gaps may not appear within the composite image. For example, a first image may correspond to a first optical region; a second image may correspond to a second optical region; and a third image may correspond to a third optical region. In this example, each region may be overlapped by at least one other region. Continuing with the example, the three example images may be gathered, and the overlap regions may be averaged or homogenized for clarity and continuity thereby helping to create a single contiguous image of all three regions based on the three individual images. In these or other embodiments, images (whether individual images or composite images) may be stored in a storage device coupled to the optical imaging device. In these or other embodiments, more or fewer than three images may comprise a composite image.

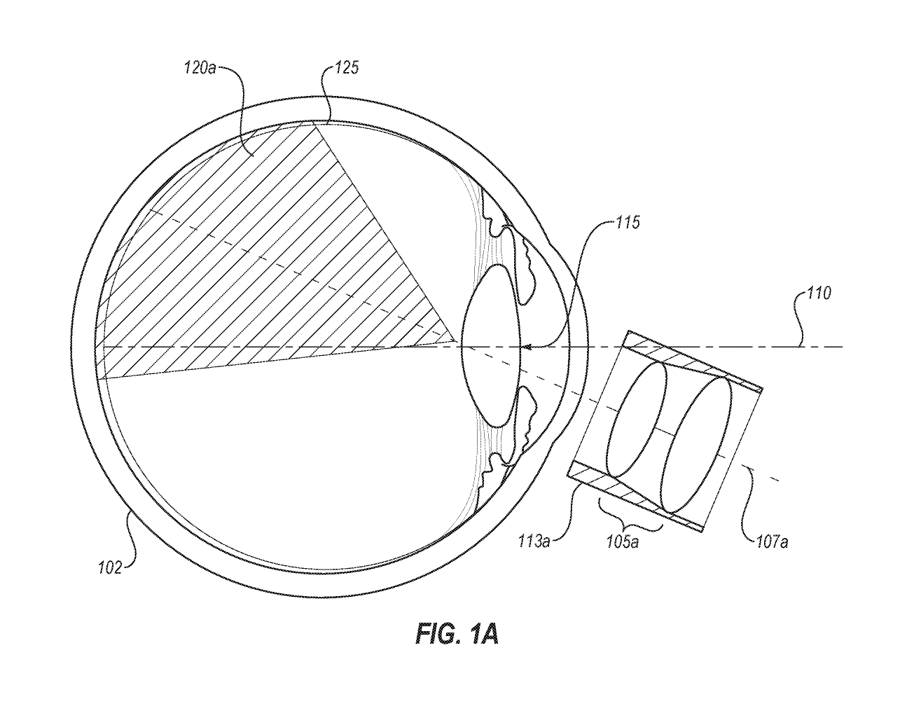

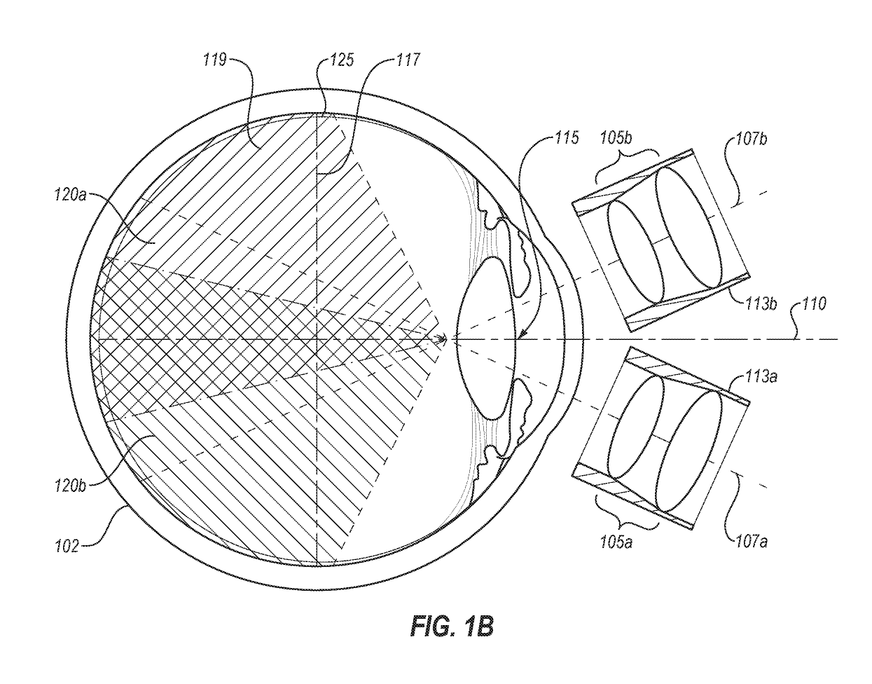

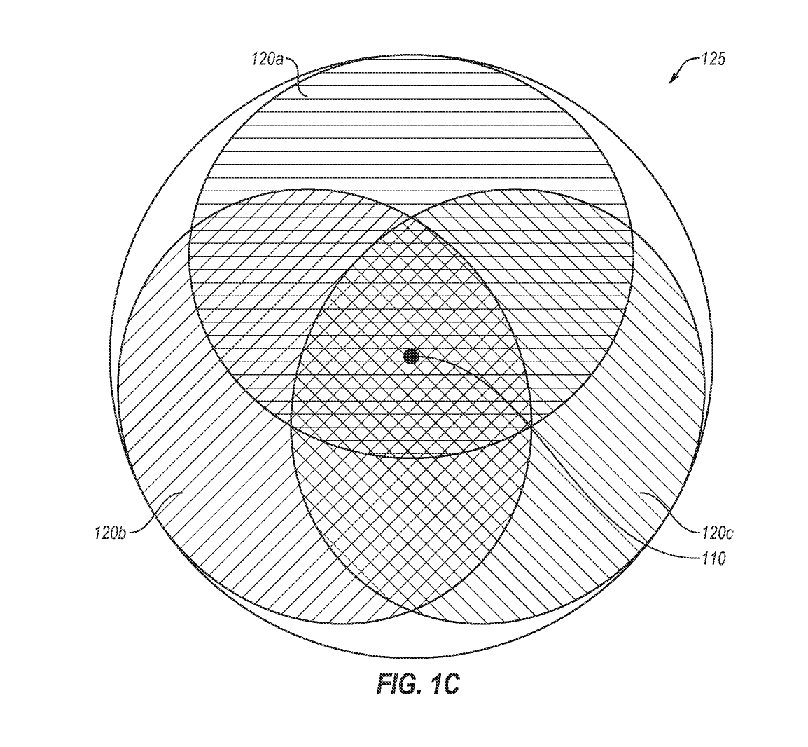

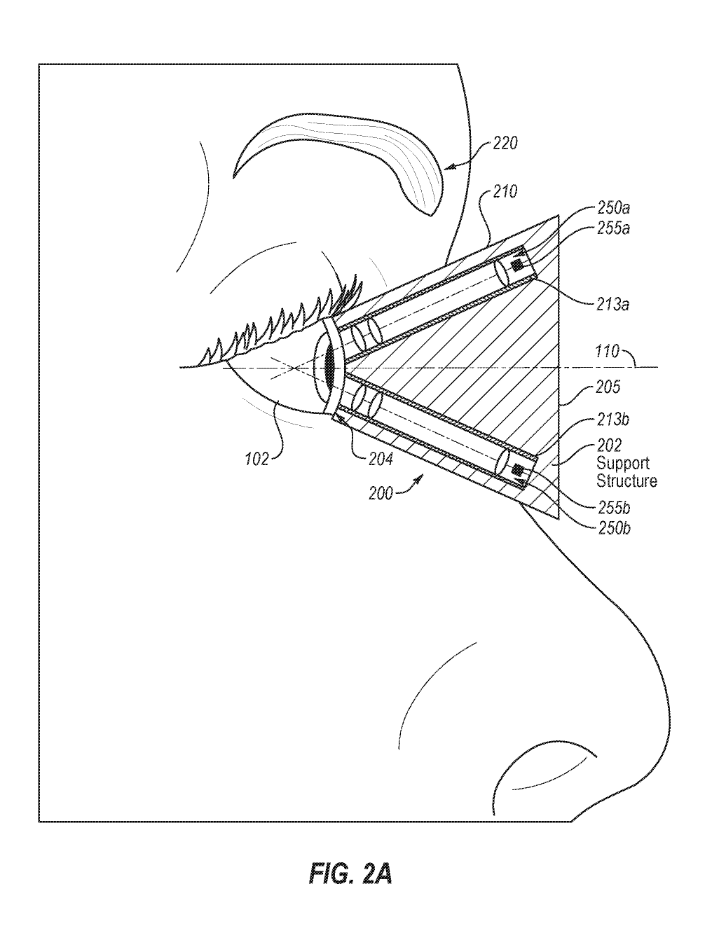

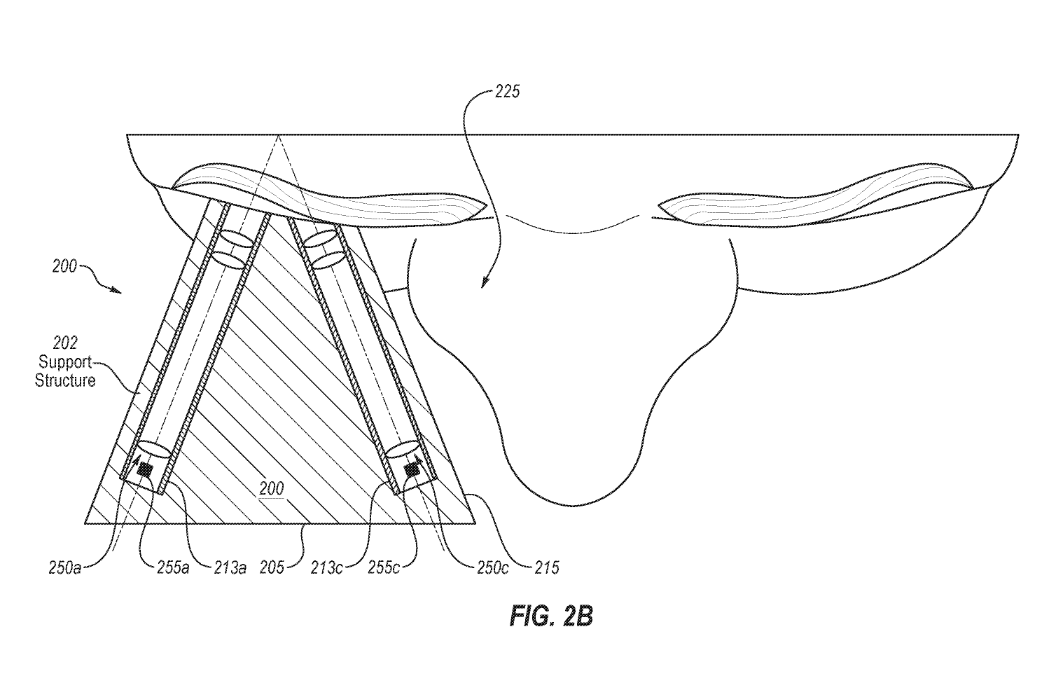

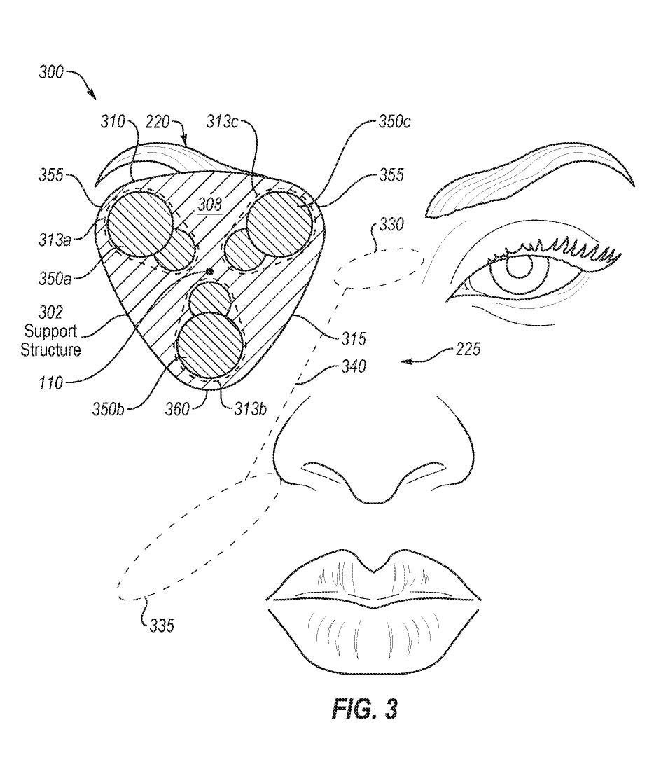

FIGS. 1A-1C indicate an example progression for achieving the composite optical image. For example, FIG. 1A illustrates a cross-sectional side view of an eye 102, including an example optical imaging pathway 107a for imaging the eye 102. FIG. 1B illustrates the same cross-sectional side view of the eye 102 with the addition of a second example optical imaging pathway 107b for imaging the eye 102. FIG. 1C illustrates three overlapping imaging regions 120a, 120b, and 120c for imaging an example area of the eye 102, including a retina 125. FIG. 1A also illustrates an imaging channel 113a, an eye lens 115, optical lenses 105a, a central axis 110, and an imaging region 120a. In these or other embodiments, the optical imaging pathway 107a may proceed from within the imaging channel 113a of a device (such as the device 200/300 illustrated in FIGS. 2A/2B and FIG. 3), through the pupil and the eye lens 115, and to the retina 125. Additionally or alternatively, the optical imaging pathway 107a may start and/or end at an image capturing device (such as a camera sensor 255 of FIGS. 2A-2B), and the image capturing device may be positioned anywhere within the imaging channel 113a. For example, the imaging capturing device may be positioned between the optical lenses 105a, along a central axis of the imaging channel 113a normal to the eye 102, and/or off the central axis of the imaging channel 113a normal to the eye 102. In these or other embodiments, the optical imaging pathway 107a may be a center axis of a field of view of the image capturing device (such as the camera sensor 255 of FIGS. 2A-2B).

Additionally or alternatively, the imaging region 120a may correspond to the optical imaging pathway 107a. For example, an area of the retina 125 that is covered by or is adjacent to the optical imaging pathway 107a may define the metes and bounds of the imaging region 120a. In other embodiments, other areas of the eye 102, such as the cornea, the iris, the iridocorneal angle, the sclera, and any other suitable area of the eye 102, whether in the anterior or posterior chamber of the eye 102, may be imaged.

In some embodiments, the optical lenses 105a may be housed by the imaging channel 113a and may collimate illumination light proceeding through the imaging channel 113a such that the illumination light proceeds collinear with and/or parallel to the optical imaging pathway 107a and illuminates at least a portion of the imaging region 120a. In some embodiments, the optical lenses 105a may be sized and shaped to fill an inner diameter of the imaging channel 113a that houses the optical lenses 105a, while in other embodiments, the optical lenses 105a may be sized and shaped to be less than the inner diameter of the imaging channel 113a. Additionally or alternatively, the optical lenses 105a may focus, disperse, and/or otherwise alter light transmission to enhance imaging capability of an image capturing device (such as the camera sensor 255 of FIGS. 2A-2B) to image the imaging region 120a.

In some embodiments, other optical elements may also be included within the imaging channel 113a. For example, a prism may be positioned anywhere within the imaging channel 113a, e.g., between the optical lenses 105a, at a distal end of the imaging channel 113a and/or at a proximal end of the imaging channel positioned between the eye 102 and the optical lenses 105a. In some embodiments, the prism may be configured as a mirror, beam splitter, or other suitable reflective element (e.g., partially reflective, substantially reflective, or completely reflective). In these or other embodiments, multiple prisms may be positioned within the imaging channel 113a, while in other embodiments, only a single prism within the imaging channel 113a. In some embodiments, the prism may help direct light to and/or from the eye 102, e.g., permitting multi-directional travel of optical signals between the eye 102 and an optical imaging device. For example, the prism may at least partially direct one or both of the optical imaging pathway 107a and an optical illumination pathway toward the eye 102.

In some embodiments, the optical imaging pathway 107a may not be coaxial to the central axis 110 of the eye 102. In this manner, multiple optical imaging pathways 107 (such as the optical imaging pathways 107a and 107b as shown in FIG. 1B) may be permitted to image the retina 125 and/or other areas of the eye 102, such as the cornea, the iris, the iridocorneal angle, the sclera, and any other suitable area of the eye 102, whether in the anterior or posterior chamber of the eye 102.

Additionally or alternatively, the optical lenses 105a may have fixed or variable positions within the imaging channel 113a. For example, one or more of the optical lenses 105a may be positionally fixed such that the optical lenses 105a may not move within the imaging channel 113a. As another example, one or more of the optical lenses 105a may be positionally movable within the imaging channel 113a such that the lenses can slide closer to the eye 102 during examination or slide farther away from the eye 102 during examination. Additionally or alternatively, the optical lenses 105a may be positionally movable within the imaging channel 113a such that the lenses can slide laterally so as to maintain a relative distance between the optical lenses 105a and the eye 102 during examination or image acquisition.

FIG. 1B illustrates another cross-sectional side view of the eye 102 of FIG. 1A, including multiple example optical imaging pathways 107 (such as the optical imaging pathways 107a and 107b) for imaging the retina 125 and/or other areas of the eye 102, such as the cornea, the iris, the iridocorneal angle, the sclera, and any other suitable area of the eye 102, whether in the anterior chamber or a posterior cavity 119 of the eye 102. Specifically, FIG. 1B shows the addition of an imaging channel 113b, an optical imaging pathway 107b, optical lenses 105b, and overlapping imaging regions 120a/120b. The imaging channel 113b, the optical imaging pathway 107b, and the optical lenses 105b may be the same as or similar to the imaging channel 113a, the optical imaging pathway 107a, and the optical lenses 105a, respectively, of FIG. 1A. Additionally or alternatively, the imaging channel 113b and/or the optical imaging pathway 107b may not be coaxial to the central axis 110 of the eye 102. Thus, in some embodiments, the optical imaging pathways 107 of the imaging channels 113 may be angled relative to each other and/or to the central axis 110. For example, in some embodiments, the optical imaging pathways 107 may cross each other at a position within the posterior cavity 119 of the eye 102, and at a position anterior to an equatorial line 117, e.g., when imaging the retina 125. In other embodiments, depending on the desired target area of the eye 102 to be imaged, such as a surface of the cornea, the iris, the iridocorneal angle or the sclera, the optical imaging pathways 107 may converge at a position in the anterior chamber or at a position anterior to an outer surface of the cornea. In other embodiments, depending on the desired target area of the eye 102 to be imaged, the optical imaging pathways 107 may converge at a position in the posterior cavity 119 of the eye 102, and at a position posterior to an equatorial line 117.

In these or other embodiments, the imaging region 120a may correspond to the optical imaging pathway 107a, and the imaging region 120b may correspond to the optical imaging pathway 107b. The imaging regions 120a/120b may include portions of, for example, the retina 125 that are captured in digital images. Additionally or alternatively, the imaging region 120a and the imaging region 120b may overlap, for example, such that one or more portions of the retina 125 are captured in both images captured through the imaging channels 113a and 113b.

In some embodiments, the imaging channels 113 may be fixed relative to each other, exactly or approximately, in terms of position in three-dimensional space or in terms of angles relative to a central optical axis of each imaging channel or the central axis 110 of the eye 102. For example, the imaging channels 113 may be angled at approximately equal angles off of the central optical axis of each imaging channel 113. Additionally or alternatively, the imaging channels 113 may be angled at approximately equal angles off of the central axis 110 of the eye 102 of the patient such that the imaging channels 113 may be evenly spaced in the 360 degrees around the central axis 110 of the eye 102 (e.g., each imaging channel 113 offset by approximately 30 degrees to approximately 45 degrees from the central axis 110 of the eye 102 and/or distributed approximately 120 degrees relative to each other).

In some embodiments, the angles between the imaging channels 113 relative to the central optical axis of each imaging channel 113 or relative to the central axis 110 of the eye 102 may not be equal or consistent. For example, different angles may accommodate different configurations and shapes of facial structures (e.g., a triangular base other than an equilateral triangle may be incorporated). In these or other embodiments, various configurations and numbers of imaging channels 113 may be used. For example, in some embodiments, a number of imaging channels 113 in the optical imaging device 200/300 (not shown) may be two, three, four or five, while in other embodiments, between six and ten imaging channels 113 may be used, while in still other embodiments, more than ten imaging channels 113 may be used.

In some embodiments, the known relative positioning of the multiple imaging channels 113 may facilitate the stitching of multiple images into a composite image via software analytics. Thus, according to some embodiments, regardless of the angles (equal or not) of the imaging channels 113 relative to the central axis 110 of the eye 102 or relative to the central optical axis of each imaging channel, the angles may be known variables to the software such that image stitching can be achieved with sufficient precision. The multiple images to be stitched into a composite image, which are obtained via image capturing devices within the imaging channels 113, may be stored in a storage device.

FIG. 1C illustrates a cross-sectional front view of the eye 102 of FIG. 1A, including multiple overlapping imaging regions 120a/120b/120c of the retina 125. In other embodiments, the multiple overlapping imaging regions 120a/120b/120c may correspond to other areas of the eye 102, such as the cornea, the iris, the iridocorneal angle, the sclera, and any other suitable area of the eye 102, whether in the anterior chamber or a posterior cavity 119 of the eye 102. With the three different but overlapping imaging regions 120a/120b/120c of, for example, the retina 125, a composite image may be obtained that includes a combined area with a greater field of view than any single imaging region 120 and without any gaps within the composite image area. In some embodiments, the central axis 110 of the eye 102 may intersect a position on the retina 125 that is within two or more of the imaging regions 120.

In these or other embodiments of the present disclosure, an optical imaging device (such as that shown in FIGS. 2A, 2B, and 3) includes an upside-down pyramidal configuration of imaging channels 113 for increasing a clearance distance relative to facial structures of patients.

Modifications, additions, or omissions may be made to the embodiments of FIGS. 1A-1C without departing from the scope of the present disclosure. For example, in some embodiments, the channels 113a/113b may include any number of other components that may not be explicitly illustrated or described. Additionally or alternatively, for example, the imaging regions 120a/120b/120c may include different sizes, shapes, overlapping areas, etc. than may be explicitly illustrated or described.

FIGS. 2A-2B illustrate a cross-sectional side and top view, respectively, of an optical imaging device 200 for imaging the eye 102, relative to facial features and arranged according to one or more embodiments of the present disclosure. As illustrated, the optical imaging device 200 may include a support structure 202, a peak 204, a front face 205, a top face 210, a side face 215, imaging channels 213 (including 213a/213b in FIG. 2A and 213a/213c in FIG. 2B), optical lenses 250 (including 250a/250b in FIG. 2A and 250a/250c in FIG. 2B), and camera sensors 255 (including 255a/255b in FIG. 2A and 255a/255c in FIG. 2B). The imaging channels 213 may be the same as or similar to the imaging channel 113 of FIGS. 1A-1B and the imaging channel 313 of FIG. 3 discussed below. Additionally or alternatively, the optical lenses 250 may be the same as or similar to the optical lenses 105a/105b of FIGS. 1A-1B.

In some embodiments, the support structure 202 may house one or more components of the optical imaging device 200, such as the imaging channels 213, the optical lenses 250, and the camera sensors 255. The camera sensors 255 may be image capturing devices that may respectively include an entire imaging sensor or a portion of a larger digital camera, where the larger digital camera may be positioned outside of the optical imaging device 200. Additionally or alternatively, the support structure 202 may be sized and shaped based on one or more facial features of a patient, e.g., a nose 225 (shown in FIG. 2B) and a brow 220 (shown in FIG. 2A). For example, in some embodiments, the side face 215 may be angled to provide clearance between the side face 215 and the nose 225. Additionally or alternatively, the top face 210 may be angled to provide clearance between the top face 210 and the brow 220. In these or other embodiments, the front face 205 may be configured as a base of the support structure 202 positioned opposite or distal to the eye 102 during image acquisition. Additionally or alternatively, the support structure 202 may include the peak 204 opposite the front face 205 or base. In these or other embodiments, the peak 204 may be sized and shaped to contact the eye 102 or be positioned proximate to the eye 102 (e.g., sized and shaped to fit general contour(s) of the eye 102 in a concentric or approximately concentric manner) during image acquisition. In these or other embodiments, the term “proximate” as referred to herein may include a distance that permits the optical imaging device 200 to be close enough to image the eye 102, for example, ranging from direct contact (zero mm) to a threshold distance of 10 mm, written as a closed range of [0,10] mm. In these or other embodiments, the imaging channels 213 may span all or a part of the region of the support structure 202 between the front face 205 and the peak 204.

Modifications, additions, or omissions may be made to the embodiments of FIGS. 2A-2B without departing from the scope of the present disclosure. For example, in some embodiments, the support structure 202 may include any number of other components that may not be explicitly illustrated or described. Additionally or alternatively, the support structure 202 may be sized, shaped, and/or oriented relative to facial features in other suitable ways than may be explicitly illustrated or described. Additionally or alternatively, for example, the imaging channels 213a/213b/213c may be sized, shaped, positioned, and/or oriented within the support structure 202 in other suitable ways than may be explicitly illustrated or described.

FIG. 3 illustrates a cross-sectional front view of an optical imaging device 300 for imaging the eye, relative to facial features. The optical imaging device 300 may be the same as or similar to the optical imaging device 200 in FIGS. 2A-2B. As illustrated, the optical imaging device 300 may include a support structure 302, a front face or base 305, a top face 310, imaging channels 313a-313c, a side face 315, optical lenses 350a-350c, base corners 355, and an apex corner 360. Additionally, the facial features illustrated include the central axis 110 of the eye 102 in FIGS. 1A-2B, the brow 220 and the nose 225 of FIGS. 2A-2B, along with a root 330 of the nose 225 and a nasolabial sulcus 335. In these or other embodiments, the optical imaging device 300 illustrates an example upside-down pyramidal configuration with the three example imaging channels 313a-313c. In some embodiments, the upside-down pyramidal configuration of the imaging channels 313 may be oriented to increase clearance between the support structure 302 and both the brow 220 and the nose 225 of a patient.

In some embodiments, the optical imaging device may approach the eye with multiple imaging channels 313 (for example, three imaging channels 313). In some embodiments, the number of imaging channels 313 may be two, three, four, five, while other embodiments may include six to ten imaging channels 313, and still other embodiments more than ten imaging channels 313. The multiple imaging channels 313 may be oriented non-coaxial to the central axis 110 of the eye and cross each other within the posterior cavity of the eye, e.g., between the equatorial line 117 and the eye lens 115 of FIG. 1B. In other embodiments, at least one imaging channel 313 may be coaxial with the central axis 110 of the eye. Additionally or alternatively, the multiple imaging channels 313 may approximately define a pyramidal cone shape near the eye, as shown for example in FIG. 3. In these or other embodiments, the optical imaging device 300 may include for the base 305, an approximately flat, triangular base 305 with rounded corners 355/360 positioned farthest away from the eye of the patient.

In some embodiments, the imaging channels 313 may be respectively positioned adjacent to the corners 355/360. For example, the imaging channel 313a may be positioned adjacent to one of the base corners 355, and the imaging channel 313c may be positioned adjacent to the other of the base corners 355. Additionally or alternatively, the imaging channel 313b may be positioned adjacent to the apex corner 360. In these or other embodiments, the optical imaging device 300 may be oriented such that the two base corners 355 are positioned above the central axis 110 of the eye, and the apex corner 360 is positioned below the central axis 110 of the eye. Additionally or alternatively, other configurations and orientations may be suitable. For example, the optical imaging device 300 may be rotated to more suitably conform to facial features of a patient, to permit image acquisition in limited pupil dilation scenarios, and other circumstances that may benefit from orientation variations of the optical imaging device 300.

Additionally or alternatively, the top face 310 of the optical imaging device 300 may be oriented approximately parallel to the brow 220 of the patient. The top face 310 may be positioned between the base 305 and the eye of the patient. Additionally or alternatively, the side face 315 of the optical imaging device may be oriented approximately parallel to the nose 225 of the patient, for example, approximately parallel to a virtual line 340 connecting the root 330 (radix) of the nose 225 and the nasolabial sulcus 335. The side face 315 may also be positioned between the base 305 and the eye of the patient.

This upside-down pyramidal configuration may increase physical clearance between the optical imaging device 300 and the brow 220, while also potentially increasing physical clearance between the optical imaging device 300 and the nose 225 and adjacent features. In some embodiments, increased clearance between the optical imaging device 300 and the facial structures of the patient may improve visibility for the user of the optical imaging device 300 during operation as well as improve physical comfort and access for patients with a wide range of facial structures. In other embodiments, additional configurations of the support structure 302, other than an upside-down pyramidal configuration, may be implemented. For example, any suitable configuration permitting additional or increased clearance between the support structure 302 and one or both of the brow 220 and the nose 225 is contemplated herein. Additionally or alternatively, any suitable configuration permitting multiple imaging channels 313, e.g., two or more imaging channels 313, for imaging the eye may be implemented.

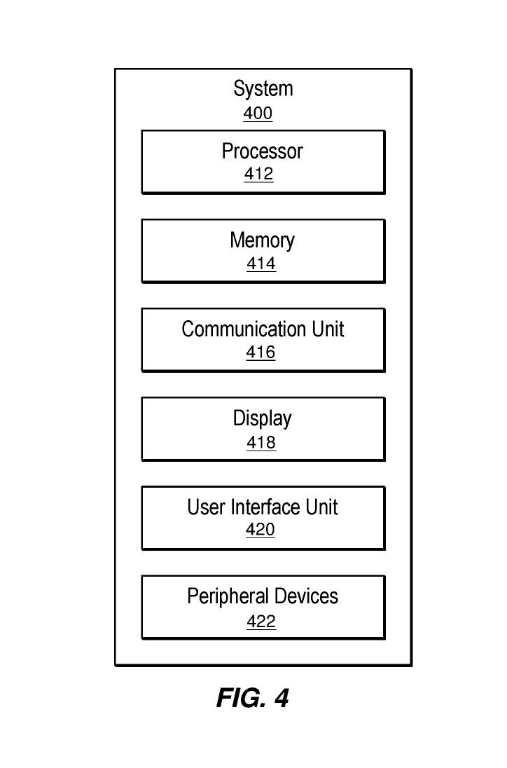

In some embodiments, the optical imaging device 300 may include or be communicatively coupled to a computing device, for example the system 400 of FIG. 4. In some embodiments, the computing device may include memory and at least one processor, which are configured to perform operations as described in this disclosure, among other operations. In some embodiments, the computing device may include computer-readable instructions that are configured to be executed by the optical imaging device 300 to perform operations described in this disclosure, e.g., to acquire optical images via the camera sensors 255 of FIGS. 2A-2B. Other example operations may include image stitching, software analytics, and other suitable operations, e.g., obtaining images to be stitched into a composite image from a storage device.

Modifications, additions, or omissions may be made to the embodiments of FIG. 3 without departing from the scope of the present disclosure. For example, in some embodiments, the support structure 302 may include any number of other components that may not be explicitly illustrated or described. Additionally or alternatively, the support structure 302 may be sized, shaped, and/or oriented relative to facial features in other suitable ways than may be explicitly illustrated or described. Additionally or alternatively, for example, the imaging channels 313a/313b/313c may be sized, shaped, positioned, and/or oriented within the support structure 302 in other suitable ways than may be explicitly illustrated or described.

FIG. 4 illustrates an example system 400 that may be used in multiple off-axis channel imaging of the eye. The system 400 may be arranged in accordance with at least one embodiment described in the present disclosure. The system 400 may include a processor 410, memory 412, a communication unit 416, a display 418, a user interface unit 420, and a peripheral device 422, which all may be communicatively coupled. In some embodiments, the system 400 may be part of any of the systems or devices described in this disclosure.

Generally, the processor 410 may include any suitable special-purpose or general-purpose computer, computing entity, or processing device including various computer hardware or software modules and may be configured to execute instructions stored on any applicable computer-readable storage media. For example, the processor 410 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data.

Although illustrated as a single processor in FIG. 4, it is understood that the processor 410 may include any number of processors distributed across any number of networks or physical locations that are configured to perform individually or collectively any number of operations described in this disclosure. In some embodiments, the processor 410 may interpret and/or execute program instructions and/or process data stored in the memory 412. In some embodiments, the processor 410 may execute the program instructions stored in the memory 412.

For example, in some embodiments, the processor 410 may execute program instructions stored in the memory 412 that are related to determining whether generated sensory data indicates an event and/or determining whether the event is sufficient to determine that the user is viewing a display of a device such that the system 400 may perform or direct the performance of the operations associated therewith as directed by the instructions. In these and other embodiments, instructions may be used to perform one or more operations or functions described in the present disclosure.

The memory 412 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 410. By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 410 to perform a certain operation or group of operations as described in this disclosure. In these and other embodiments, the term “non-transitory” as explained in the present disclosure should be construed to exclude only those types of transitory media that were found to fall outside the scope of patentable subject matter in the Federal Circuit decision of In re Nuijten, 500 F.3d 1346 (Fed. Cir. 2007). Combinations of the above may also be included within the scope of computer-readable media.

The communication unit 416 may include any component, device, system, or combination thereof that is configured to transmit or receive information over a network. In some embodiments, the communication unit 416 may communicate with other devices at other locations, the same location, or even other components within the same system. For example, the communication unit 416 may include a modem, a network card (wireless or wired), an infrared communication device, a wireless communication device (such as an antenna), and/or chipset (such as a Bluetooth device, an 802.6 device (e.g., Metropolitan Area Network (MAN)), a Wi-Fi device, a WiMax device, cellular communication facilities, etc.), and/or the like. The communication unit 416 may permit data to be exchanged with a network and/or any other devices or systems described in the present disclosure.

The display 418 may be configured as one or more displays, like an LCD, LED, or other type of display. For example, the display 418 may be configured to present measurements, indicate warning notices, show tolerance ranges, display whether good/bad eye tissues are determined, and other data as directed by the processor 410.

The user interface unit 420 may include any device to allow a user to interface with the system 400. For example, the user interface unit 420 may include a mouse, a track pad, a keyboard, buttons, and/or a touchscreen, among other devices. The user interface unit 420 may receive input from a user and provide the input to the processor 410. In some embodiments, the user interface unit 420 and the display 418 may be combined.

The peripheral devices 422 may include one or more devices. For example, the peripheral devices may include a sensor, a microphone, and/or a speaker, among other peripheral devices. As examples, the sensor may be configured to sense changes in light, sound, motion, rotation, position, orientation, magnetization, acceleration, tilt, vibration, etc., e.g., as relating to an eye of a patient. Additionally or alternatively, the sensor may be part of or communicatively coupled to the optical imaging device as described in the present disclosure.

Modifications, additions, or omissions may be made to the system 400 without departing from the scope of the present disclosure. For example, in some embodiments, the system 400 may include any number of other components that may not be explicitly illustrated or described. Further, depending on certain implementations, the system 400 may not include one or more of the components illustrated and described.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner. Additionally, the terms “about,” “substantially,” and “approximately” should be interpreted to mean a value within 10% of an actual value, for example, values like 3 mm or 100% (percent).

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. An optical imaging device, comprising:
a support structure; and
a plurality of imaging channels, each imaging channel of the plurality of imaging channels including a discrete optical imaging pathway, the plurality of imaging channels disposed within the support structure, the plurality of imaging channels aimed at different angles relative to each other such that each optical imaging pathway is directed through a pupil of the eye towards corresponding partially overlapping regions of a retina.

2. The optical imaging device of claim 1, further comprising a plurality of image capturing devices, each image capturing device of the plurality of image capturing devices respectively associated with one of the plurality of imaging channels to capture digital photograph images of respective portions of the eye.

3. The optical imaging device of claim 2, wherein the digital photograph images overlap each other and are stored in a storage device of the optical imaging device for stitching together such that the digital photograph images form a composite image.

4. The optical imaging device of claim 1, wherein the plurality of imaging channels form a pyramidal configuration oriented to increase clearance between the support structure and both a brow structure and a nasal structure of a patient.

5. The optical imaging device of claim 4, wherein the pyramidal configuration includes a base at a bottom end of the pyramidal configuration and a peak positioned opposite to the base at a top end of the pyramidal configuration, the peak configured to be proximate to the eye when the optical imaging device images the eye.

6. The optical imaging device of claim 5, wherein the peak is sized and shaped to contact the eye when the optical imaging device images the eye.

7. The optical imaging device of claim 5, wherein the plurality of imaging channels span between the base and the peak.

8. The optical imaging device of claim 5, wherein:
the base is shaped with three corners including two base corners and an apex corner; and
the plurality of imaging channels includes three imaging channels, two of the three imaging channels being respectively positioned at the two base corners and one of the three imaging channels being positioned at the apex corner.

9. The optical imaging device of claim 8, wherein the two base corners are positioned above a center axis of the support structure and the apex corner is positioned below the center axis of the support structure, the center axis of the support structure configured to be collinear with the central axis of the eye when the optical imaging device images the eye.

10. The optical imaging device of claim 5, further comprising:
an image capturing device positioned within each imaging channel of the plurality of imaging channels that corresponds respectively to one or more optical lenses within each imaging channel of the plurality of imaging channels.

11. The optical imaging device of claim 1, further comprising:
a plurality of sets of optical lenses, at least one lens in each of the sets of optical lenses having a fixed position or a variable position within a respective imaging channel of the plurality of imaging channels.

12. The optical imaging device of claim 1, wherein the discrete optical imaging pathways of the plurality of imaging channels converge at a position inside a posterior cavity of the eye and anterior to an equatorial line of the eye.

13. A system comprising:
one or more processors configured to receive optical imaging data; and
an optical imaging device configured to generate optical imaging data, the optical imaging device communicatively coupled to the one or more processors, and the optical imaging device comprising:
a support structure;
a plurality of imaging channels, each imaging channel of the plurality of imaging channels including a discrete optical imaging pathway, the plurality of imaging channels disposed within the support structure, the plurality of imaging channels aimed at different angles relative to each other such that each optical imaging pathway is directed through the pupil of the eye towards corresponding partially overlapping regions of a retina;
one or more optical lenses within each imaging channel of the plurality of imaging channels; and
a plurality of image capturing devices, at least one image capturing device of the plurality of image capturing devices positioned within each imaging channel of the plurality of imaging channels that corresponds respectively to the one or more optical lenses within each imaging channel of the plurality of imaging channels.

14. The system of claim 13, wherein:
each of the image capturing devices respectively positioned within each imaging channel of the plurality of imaging channels captures digital photograph images of respective portions of the eye; and
the digital photograph images overlap each other and are stored in a storage device of the optical imaging device for stitching together such that the digital photograph images form a composite image.

15. The system of claim 13, wherein the plurality of imaging channels form a pyramidal configuration oriented to increase clearance between the support structure and both a brow structure and a nasal structure of a patient.

16. The system of claim 15, wherein:
the pyramidal configuration includes a base at a bottom end of the pyramidal configuration and a peak positioned opposite to the base at a top end of the pyramidal configuration, the peak configured to be proximate to the eye when the optical imaging device images the eye; and
the plurality of imaging channels span between the base and the peak.

17. The system of claim 16, wherein the peak is sized and shaped to contact the eye when the optical imaging device images the eye.

18. The system of claim 16, wherein:
the base is shaped with three corners including two base corners and an apex corner;
the plurality of imaging channels includes three imaging channels, two of the three imaging channels being respectively positioned at the two base corners and one of the three imaging channels being positioned at the apex corner; and
the two base corners are positioned above a center axis of the support structure and the apex corner is positioned below the center axis of the support structure, the center axis of the support structure configured to be collinear with the central axis of the eye when the optical imaging device images the eye.

19. The system of claim 13, further comprising:
a plurality of sets of optical lenses, at least one lens in each of the sets of optical lenses having a fixed position or a variable position within a respective imaging channel of the plurality of imaging channels.

20. The system of claim 13, wherein the discrete optical imaging pathways of the plurality of imaging channels converge at a position inside a posterior cavity of the eye and anterior to an equatorial line of the eye.
